(19) United States
(12) Patent Application Publication
(10) Pub. No.: US 2022/0386624 A1
(43) Pub. Date: Dec. 8, 2022

(54) STRAIN OF PSEUDOMONAS AERUGINOSA 9# AND APPLICATIONS THEREOF

(71) Applicant: China National Rice Research Institute, Hangzhou (CN)

(72) Inventors: Xiaoyan LIN, Hangzhou (CN); Mingxue CHEN, Hangzhou (CN); Zhaoyun CAO, Hangzhou (CN); Huan YANG, Hangzhou (CN)

(21) Appl. No.: 17/887,538

(22) Filed: Aug. 15, 2022

(51) Int. Cl. A01N 63/27; C12N 1/20; A01P 3/00

(52) U.S. Cl. CPC A01N 63/27; C12N 1/205; A01P 3/00; C12R 2001/385

(57) ABSTRACT

A strain of Pseudomonas aeruginosa 9 # and its applications are disclosed, which relates to the technical field of microorganisms. The deposit number of the Pseudomonas aeruginosa 9 # of the disclosure is CCTCC NO: M 2021178. The strain of Pseudomonas aeruginosa 9 # can inhibit the growth of the Ustilaginodea vixens strain YY7850, which is the first instance that the Pseudomonas aeruginosa strain has been found to antagonize the pathogen of rice false smut. In view of the ability of strain 9 # to inhibit the pathogen of rice false smut, the strain has the potential to be developed into a biocontrol bacterium of rice false smut.

### TECHNICAL FIELD

The present disclosure relates to the technical field of microorganisms, and more specifically, to a strain of Pseudomonas aeruginosa 9 # and its applications.

### BACKGROUND ART

Rice false smut is a fungal disease of rice panicle caused by Ustilaginodea virens (Cooke) Takahashi, which infects spikelets, glumes and grains at the late stage of rice growth, commonly known as “harvest disease”. Rice false smut is a worldwide rice disease, which occurs in China, Japan, the United States, India, the Philippines and other countries. In recent years, it has been upgraded from a minor disease on rice to one of the major diseases. The pathogen consumes a large amount of nutrients from diseased grains in the process of reproduction, which not only endangers the grain itself where rice false smut occurs, but also affects the nutrient transport of adjacent grains, resulting in an increase in the sterile grain percentage and a decrease in the seed setting rate and 1000-grain weight, resulting in a reduction in yield. Rice false smut forms rice smut in the panicle of rice, which seriously affects the growth of grains and reduces the yield and quality of rice, and the toxin of rice false smut produced by its pathogenic fungus Ustilaginodea virens (Cooke) Takahashi is toxic to humans and animals At present, the fungus producing rice false smut and the toxins produced by rice false smut have gradually attracted extensive attention of experts and scholars.

In natural ecosystems, pathogens may have many “natural enemies” or “competitors”, so microorganisms that can kill or inhibit pathogens have potential biological control value. Antagonistic bacteria can be widely used in the control of many diseases and pests. Microbial antagonists can directly produce antibacterial effects through parasitism, antibiosis or competition, and can also indirectly protect plants by inducing their own resistance. It is a new measure with sustainable development potential to extract antibacterial substances that have antagonistic activity against pathogenic bacteria, or screen microorganisms that have antagonistic activity against pathogenic bacteria for biological control. The biocontrol bacteria may come from the plant body or from the relevant living environment.

Therefore, it is an urgent problem for those skilled in the art to provide a strain of Pseudomonas aeruginosa 9 # and its applications.

### SUMMARY

In view of the above, the disclosure provides a strain of Pseudomonas aeruginosa 9 # and its applications. Pseudomonas aeruginosa 9 is used to antagonize Ustilaginodea virens (Cooke) Takahashi and control rice false smut.

In order to achieve the above purpose, technical solutions of the present disclosure are specifically described as follows.

A strain of Pseudomonas aeruginosa 9 #, with the deposit number of CCTCC NO:M 2021178, has been deposited in CHINA CENTER FOR TYPE CULTURE COLLECTION, referred to as CCTCC, and the address is Wuhan University, Wuhan, China. The deposit date is Feb. 2, 2021. And the classification name is Pseudomonas aeruginosa 9 #.

Further, an application of the strain of Pseudomonas aeruginosa 9 # in antagonizing Ustilaginodea virens (Cooke) Takahashi is provided.

Further, an application of the strain of Pseudomonas aeruginosa 9 # in preventing and treating rice false smut is provided.

Further, an application of the strain of Pseudomonas aeruginosa 9 # in a preparation of microbial agents for preventing and treating rice false smut is provided.

According to the above technical solutions, compared with the prior art, a strain of Pseudomonas aeruginosa 9 # and its applications are provided. The strain of Pseudomonas aeruginosa 9 # can inhibit the growth of the Ustilaginodea virens strain YY7850, which is the first instance that the Pseudomonas aeruginosa strain has been found to antagonize the pathogen of rice false smut. In view of the ability of strain 9 # to inhibit the pathogen of rice false smut, the strain has the potential to be developed into a biocontrol bacterium of rice false smut.

In addition, the strain has strong tolerance to a variety of heavy metals, such as Cd (2200 mg/L), Zn (1800 mg/L) and Pb (1200 mg/L). Among them, the strain has the strongest tolerance to Cd. The strain is considered to be a polymetallic tolerant bacterial strain. The strain is also sensitive to 13 kinds of different antibiotics. The strain is also a Cd adsorbing strain. The simulated field experiment showed that the strain can significantly reduce Cd accumulation in rice grains (Lin X Y, Mou R X, Cao Z Y, et al. Characterization of cadmium-resistant bacteria and their potential for reducing accumulation of cadmium in rice grains. Science of the Total Environment, 2016, 569-570: 97-104). Therefore, this strain is a multifunctional strain, which has great development and utilization value in the control of rice false smut disease and the microbial remediation of contaminated soil in paddy fields.

### BRIEF DESCRIPTION OF THE DRAWINGS

In order to explain the embodiments of the present disclosure or the technical solutions in the prior art more clearly, the following drawings that need to be used in the description of the embodiments or the prior art will be briefly introduced. Obviously, the drawings in the following description are only embodiments of the present disclosure. For those of ordinary skill in the art, other drawings can be obtained based on the drawings disclosed without creative work.

FIG. 1 shows the colony morphology of the strain 9 # of the disclosure.

FIG. 2 is the phylogenetic tree of the strain 9 # of the disclosure.

FIG. 3 shows the antibacterial effect of the strain 9 # of the disclosure on the Ustilaginodea virens strain YY7850.

Where, A is the control, and B is the antagonism diagram.

### DETAILED DESCRIPTION OF THE EMBODIMENTS

Technical solutions of the present disclosure will be clearly and completely described below with reference to the embodiments. Obviously, the described embodiments are only part of the embodiments of the present disclosure, not all of them. Based on the embodiments of the disclosure, all other embodiments made by those skilled in the art without sparing any creative effort should fall within the protection scope of the disclosure.

### Embodiment 1 Screening and Identification of Strain 9 #

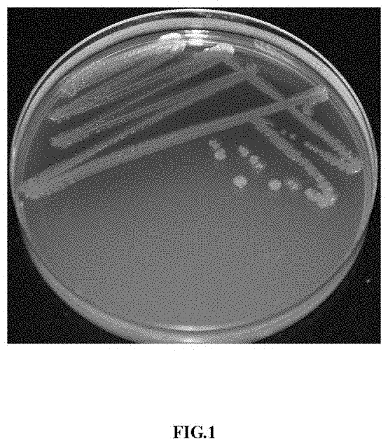

The antagonistic experiment of Ustilaginodea virens (Cooke) Takahashi was carried out on several bacterial strains isolated and purified from the soil of the experimental field of China National Rice Research Institute. A bacterial strain, named strain 9 #, was successfully screened through the antagonistic experiment. The colony morphology is shown in FIG. 1. After cultured on ordinary agar, the bacterium formed large and flat colonies, which were ground glass like, had metallic luster and ginger flavor, could produce blue-green pigment, and diffused in the culture medium.

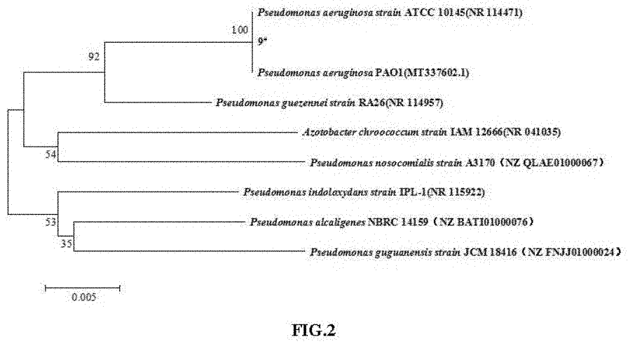

Strain 9 # was identified as a strain of Pseudomonas aeruginosa by 16s sequencing and comparative analysis. The phylogenetic tree is shown in FIG. 2.

### Embodiment 2 Antagonistic Test

(1) Strain Culture

A. Culture of Ustilaginodea Virens Strain YY7850:

The Ustilaginodea virens strain YY7850 preserved in the laboratory was inoculated on potato sucrose agar medium (PSA) plate and cultured at a constant temperature of 28° C. After the colonies grow, they are inoculated into potato sucrose liquid medium (PS), cultured at 150 rpm at a constant temperature of 28° C. for 7 days, and then filtered with eight layers of gauze to obtain spore suspension for standby.

Potato sucrose agar medium (PSA): PSA agar medium produced by Beijing Solarbio Technology Co., Ltd. was used. The PSA agar medium was prepared by adding 47.0 g PSA agar medium to each liter of water. After subpackaging, PSA agar medium was sterilized at 121° C. for 20 minutes under high pressure and damp heat. Then it was taken out and shaken well. After cooling, it was stored at 4° C. for standby.

Potato sucrose liquid medium (PS): 200 g potatoes were taken and 1000 mL of water was added. The potato was boiled with water for 20˜30 minutes, and was filtered with eight layers of gauze. 20 g sucrose was added to the filtrate, and the volume was fixed to 1 L. After high-pressure moist heat sterilization at 121° C. for 20 minutes, it was taken out and cooled for standby.

B. Culture of the Antagonistic Strain 9 #

Strain 9 # was inoculated on LB slant and cultured at a constant temperature of 37° C. for 1 day. After the colonies grew, they were inoculated into LB liquid medium, cultured at 150 rpm at a constant temperature of 37° C. for 1 day for standby.

LB slant: LB agar medium produced by Sangon Biotech (Shanghai) Co., Ltd. was used as the culture medium. LB slant was prepared by adding 35.0 g LB agar medium per liter of water. After subpackaging, it was sterilized with 121° C. high-pressure damp heat for 20 minutes, and the slant was placed while it was hot, and was stored at 4° C. for standby after the slant was cooled.

LB liquid medium: LB liquid medium produced by Sangon Biotech (Shanghai) Co., Ltd. was used. LB liquid medium was prepared by adding 35.0 g LB liquid medium per liter of water. After subpackaging, it was sterilized with 121° C. high-pressure damp heat for 20 minutes, taken out and cooled for standby.

(2) Antagonistic Test

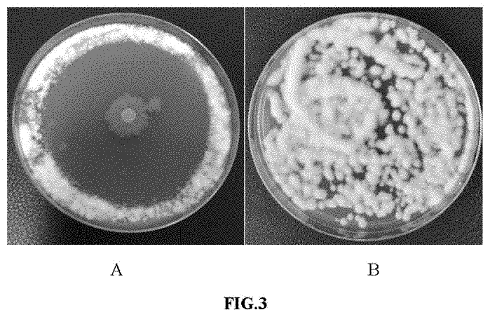

150 μL spore suspension (the spore number was 1×10⁶ cfu/mL) of Ustilaginodea virens strain YY7850 cultured in step (1) was taken and evenly coated on a sterile PSA plate with a diameter of 9 cm with a sterilized coating rod. After 28° C. constant temperature pre culture for 1 day, a sterile filter paper with a diameter of 6 mm was taken and placed on the middle of the PSA plate. 5 μL of antagonistic strain 9 # bacterial solution cultured in step (1) was added (the plate coated with only Ustilaginodea virens strain YY7850 spore suspension without antagonistic 9 # was as the control plate). It was cultured at a constant temperature of 28° C. in an upright position until the control plate was full of colonies. The radius of the antibacterial circle was measured, and the antagonistic result is shown in FIG. 3. It can be seen from FIG. 3 that strain 9 # had obvious antibacterial effect on Ustilaginodea virens strain YY7850, and the radius of the antibacterial circle was 3.3 cm. Thus, the screened strain 9 # has the ability to inhibit the pathogen of rice false smut, and has the potential to develop into a biocontrol bacterium of rice false smut.

The above description of the disclosed embodiments enables the skilled in the art to achieve or use the disclosure. Multiple modifications to these embodiments will be apparent to those skilled in the art, and the general principles defined herein may be achieved in other embodiments without departing from the spirit or scope of the disclosure. The present disclosure will therefore not be restricted to these embodiments shown herein, but rather to comply with the broadest scope consistent with the principles and novel features disclosed herein.

What is claimed is:

1. A strain of Pseudomonas aeruginosa 9 #, wherein a deposit number of the Pseudomonas aeruginosa 9 # is CCTCC NO: M 2021178.

2. An application of the strain of Pseudomonas aeruginosa 9 # of claim 1 in antagonizing Ustilaginodea vixens.

3. An application of the strain of Pseudomonas aeruginosa 9 # of claim 1 in preventing and treating rice false smut.

4. An application of the strain of Pseudomonas aeruginosa 9 # of claim 1 in a preparation of microbial agents for preventing and treating rice false smut.
